(19) United States
(12) Patent Application Publication
(10) Pub. No.: US 2016/0256551 A1
(43) Pub. Date: Sep. 8, 2016

(54) ADHD/OCD Treatment

(71) Applicant: Michael Lewis Moravitz, Fairfax, VA (US)

(72) Inventor: Michael Lewis Moravitz, Fairfax, VA (US)

(21) Appl. No.: 14/999,249

(22) Filed: Apr. 15, 2016

(51) Int. Cl. A61K 45/00

(52) U.S. Cl. CPC A61K 45/00

(57) ABSTRACT

Research should be undertaken on different causes and at different ages for Attention Deficit/Hyperactivity Disorder and Obsessive Compulsive Disorder. The causes may be chronic pain, addictions, and traumas rather than genetic.

Research should be conducted on causes of Attention Deficit/Hyperactivity Disorder and Obsessive Compulsive Disorder.

These disorders may be in people of all ages and would essentially be caused by non-genetic factors, including chronic pain, addictions, and traumas. ADHD may not be a learning disability in children at all. This theory also may involve may other classifications of psychological disorders but from similar causes and with similar symptoms. Older patients especially may suffer from a lack of concentration because of chronic pain. This would cause unease and lack of focus—an inability to pay attention to conversations, movies, television, reading material, etc. Addictions of all sorts may also cause these disorders: the patient would be thinking—even subconsciously—on the need for his/or her addictive substance or behavior. Traumas throughout life may also play a role in ADHD and OCD. The patient may focus on common tasks, work, or some other compulsive behavior to avoid troubling memories or facing psychological issues. This research could very well lead to new understandings and new treatments for patients of all ages, rather than classifying ADHD as a learning disability in children. Children, through the mother or environmental factors after birth, may be addicted to some substances they are exposed to, including illegal narcotics, tobacco, and alcohol. See for example Burger P H, Goecke T W, Fasching P A, Moll G, Heinrich H, Beckmann M W, Kornhuber J (September 2011). “[How does maternal alcohol consumption during pregnancy affect the development of attention deficit/hyperactivity syndrome in the child]” Fortschr Neurol Psychiatr (in German), and Abbott L C, Winzer-Serhan U H (April 2012). “Smoking during pregnancy: lessons learned from epidemiological studies and experimental studies using animal models”. Crit. Rev. Toxicol. This can lead to preventative treatments, such as in the father and mother, as well as new treatments for the children diagnosed with these disorders.

1) Builds on patents for psychiatric stimulants; and

2) Patents for psychotropic medicines.
